(19) United States
(12) Patent Application Publication
(10) Pub. No.: US 2023/0154616 A1
(43) Pub. Date: May 18, 2023

(54) PREDICTING PROGNOSIS IN GLIOBLASTOMA USING HISTOPATHOLOGY VIA AN END-TO-END MACHINE LEARNING PIPELINE

(71) Applicant: Case Western Reserve University, Cleveland, OH (US)

(72) Inventors: Pallavi Tiwari, Wexford, PA (US); Ruchika, Cleveland, OH (US)

(21) Appl. No.: 17/976,926

(22) Filed: Oct. 31, 2022

Related U.S. Application Data
(60) Provisional application No. 63/280,280, filed on Nov. 17, 2021.

(51) Int. Cl. G16H 50/20

(52) U.S. Cl. CPC G16H 50/20

(57) ABSTRACT

In some embodiments, the present disclosure relates to a non-transitory computer-readable medium storing computer-executable instructions that, when executed, cause a processor to perform operations, that include obtaining an imaging data set having one or more digitized images from one or more patients with glioblastoma (GBM). A machine learning pipeline is utilized to generate a prognosis using one or more machine learning features that describe a morphology of the one or more digitized images. Utilizing the machine learning pipeline includes utilizing a first machine learning stage to segment the one or more digitized images to identify one or more cellular tumor (CT) regions; and utilizing a second machine learning stage to generate one or more machine learning features that describe a morphology of the one or more CT regions and to further determine the prognosis from one or more machine learning features.

### REFERENCE TO RELATED APPLICATION

This application claims the benefit of U.S. Provisional Application No. 63/280,280, filed on Nov. 17, 2021, the contents of which are hereby incorporated by reference in their entirety.

### BACKGROUND

Glioblastoma is an aggressive type of cancer that can occur in the brain and/or spinal cord. Glioblastoma forms from cells called astrocytes, which support nerve cells. Glioblastoma, also known as glioblastoma multiforme, can be very difficult to treat. Furthermore, while treatments may slow tumor progression and reduce signs and symptoms, a cure is often not possible.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a flow diagram of some embodiments of a method of utilizing a machine learning pipeline to determine a prognosis for a patient having GBM (Glioblastoma).

FIG. 2 illustrates some embodiments of a block diagram corresponding to a method and/or apparatus comprising a machine learning pipeline that is configured to determine a prognosis for a patient having GBM.

FIG. 3 illustrates some additional embodiments of a block diagram corresponding to a method and/or apparatus comprising a machine learning pipeline that is configured to determine a prognosis for a patient having GBM.

FIG. 4 illustrates some additional embodiments of a block diagram corresponding to a method and/or apparatus comprising a machine learning pipeline that is configured to determine a prognosis for a patient having GBM.

FIG. 5 illustrates some embodiments of a second machine learning stage within a disclosed machine learning pipeline.

FIG. 6 illustrates a flow diagram of some embodiments of a method of utilizing a gender specific machine learning pipeline to determine a prognosis for a patient having GBM.

FIG. 7 illustrates some embodiments of a block diagram corresponding to a method and/or apparatus comprising a gender specific machine learning pipeline that is configured to determine a prognosis for a patient having GBM.

FIG. 8 illustrates some exemplary Kaplan Meier (KM) curves generated by one or more disclosed machine learning pipelines.

FIG. 9A illustrates some exemplary risk density maps generated by one or more disclosed machine learning pipelines.

FIG. 9B illustrates some exemplary t-SNE (t-distributed Stochastic Neighbor Embedding) plots generated by one or more disclosed machine learning pipelines.

FIG. 10 illustrates a flow diagram of some embodiments of a method of generating a machine learning pipeline that is configured to determine a prognosis for a patient having GBM with training and test sets.

FIG. 11 illustrates some embodiments of a block diagram corresponding to a method and/or apparatus for generating a machine learning pipeline that is configured to determine a prognosis for a patient having GBM with training and test sets.

FIG. 12 illustrates a flow diagram of some additional embodiments of a method of generating a machine learning pipeline that is configured to determine a prognosis for a patient having GBM and applying the machine learning pipeline to an additional patient.

FIG. 13 illustrates some embodiments of an apparatus comprising a machine learning pipeline that is configured to determine a prognosis for a patient having GBM.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Glioblastoma (GBM) is a highly aggressive tumor that begins within the brain and/or spinal cord. Treatment for GBM typically includes multi-modal treatment that may start with surgery, followed by radiation, and/or chemotherapy. Patients with GBM have very low survival rates. For example, despite multi-modal treatment including maximally safe surgical resection followed by radiotherapy with concomitant and adjuvant chemotherapy, GBM has a median survival of 15-18 months.

It has been appreciated that heterogeneity in GBM is able to be used to make prognostic determinations about the GBM. For example, histopathological attributes (e.g., morphological features) captured from surgically resected histopathology slides of GBM may have the ability to reveal inherent heterogeneity of the GBM and thus may have prognostic implications. Currently, visual examination of Hematoxylin and Eosin (H&E) tissue slides is the gold standard for diagnosis of GBM. However, visual identification of tumor niches within H&E tissue slides is time consuming and suffers from intra-observer and inter-observer variability. Moreover, given the high intra-tumoral heterogeneity within GBM tumors, a patient's survival information may not be visually appreciated via manual inspection of the tumor niches, even by highly experienced pathologists.

The present disclosure relates to a method for using a machine learning pipeline to generate machine learning features that describe histopathological attributes (e.g., a morphology) of cellular tumor (CT) regions on digitized images (e.g., Hematoxylin and Eosin (H&E)-stained digitized tissue slides) of surgically resected Glioblastoma (GBM) to enable prognosis (e.g., risk-stratification) of GBM. In some embodiments, the method comprises obtaining an imaging data set having one or more digitized biopsy images from a patient having GBM. The one or more digitized biopsy images are provided to a machine learning pipeline. The machine learning pipeline utilizes a first machine learning stage to segment the digitized biopsy images in a manner that identifies one or more CT regions. The machine learning pipeline further utilizes a second machine learning stage to extract one or more machine learning features describing histopathological attributes of the one or more CT regions and to determine a prognosis (e.g., an overall survival) of the patient from the identified one or more machine learning features. Thus, by utilizing the machine learning pipeline to identify the one or more CT regions, to generate and to subsequently extract the one or more machine learning features describing one or more CT regions, and to determine a patient's prognosis from the one or more machine learning features, the machine learning pipeline can make accurate prognosis of patients having GBM to enable better care for patients.

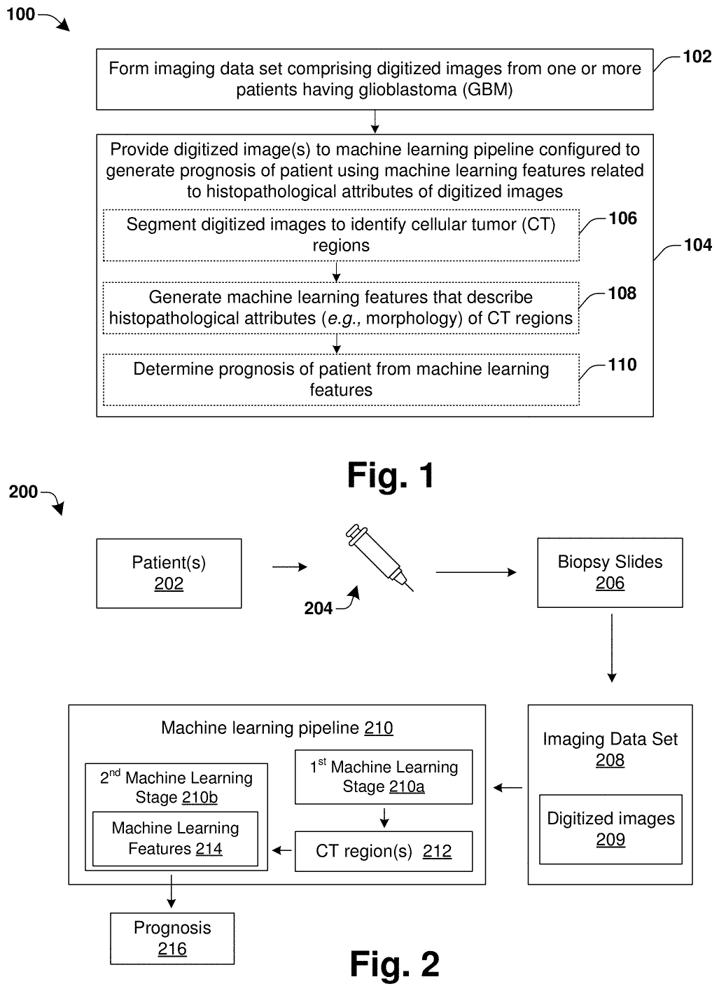

FIG. 1 illustrates a flow diagram of some embodiments of a method 100 of utilizing a machine learning pipeline to determine a prognosis for a patient having GBM.

While the disclosed methods (e.g., methods 100, 600, 1000, and/or 1200) are illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 102, an imaging data set comprising one or more digitized images are formed from one or more patients having GBM (glioblastoma). In some embodiments, the one or more digitized images may comprise digitized biopsy images. In some embodiments, the one or more digitized images may comprise digitized H&E slides from surgically resected GBM.

At act 104, the one or more digitized images are provided to a machine learning pipeline that is configured to generate a prognosis (e.g., an overall survival, a risk of cancer recurrence, etc.) of a patient using machine learning features that related to histopathological attributes (e.g., morphological features) of the digitized images. In some embodiments, the one or more digitized images may be used to train the machine learning pipeline. In some embodiments, the one or more digitized images may be provided to the machine learning pipeline after it has been trained. In some embodiments, the machine learning pipeline may be configured to generate a prognosis according to acts 106-110.

At act 106, the one or more digitized images are segmented to identify one or more cellular tumor (CT) regions within the digitized images. In some embodiments, the segmentation may separate the CT regions from background and/or necrotic regions.

At act 108, one or more machine learning features are generated from the CT regions. The one or more machine learning features describe one or more histopathological attributes (e.g., a morphology) of the one or more CT regions.

At act 110, a prognosis (e.g., an overall survival) of the patient is determined from the one or more machine learning features of CT regions. Given that phenotypic information in the one or more digitized images may reflect an aggregate effect of molecular alterations in cancer cell behavior, the one or more machine learning features can be used by the machine learning pipeline to generate a prognosis (e.g., of overall survival) in a GBM patient.

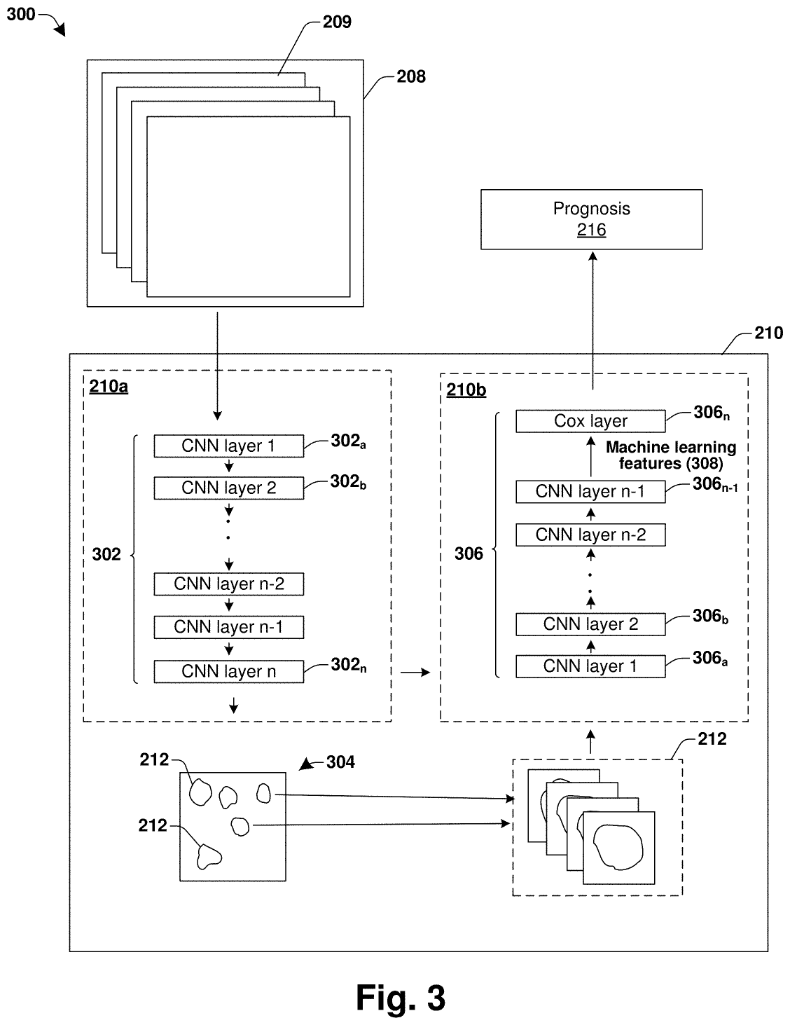

FIG. 2 illustrates some embodiments of a block diagram 200 corresponding to a method and/or apparatus comprising a machine learning pipeline that is configured to determine a prognosis for a patient having GBM.

As shown in block diagram 200, an imaging data set 208 is provided and/or formed. The imaging data set 208 comprises one or more digitized images 209 from one or more patients 202 having GBM. In some embodiments, the one or more digitized images 209 may comprise digitized biopsy images (e.g., digitized images of stained biopsy slides). For example, the one or more digitized images 209 may comprise digitized H&E (Hematoxylin and Eosin) stain images. In some such embodiments, a biopsy 204 (e.g., surgical resection) is performed on one of the one or more patients 202 to obtain a tissue block of GBM. The tissue block is sliced into thin slices that are placed on transparent slides (e.g., glass slides) to generate biopsy slides 206. The biopsy slides 206 are subsequently converted to the one or more digitized images 209 (e.g., whole slide images (WSI)).

In some embodiments, the imaging data set 208 may comprise one or more digitized images 209 having one or more cellular tumor (CT) regions on a WSI. In such embodiments, the one or more digitized images 209 comprise GBM tissue having diverse histological tumor niches in a tumor micro-environment including tumor infiltration, necrosis, pseudopalisading cells, microvascular proliferation (MVP), or the like. In some embodiments, the imaging data set 208 may comprise one or more digitized images 209 that have corresponding overall survival, censor (e.g., last known health information), biological sex, and other relevant clinical information.

The one or more digitized images 209 are provided to a machine learning pipeline 210 (e.g., a deep learning pipeline) that is configured to determine a prognosis 216 (e.g., an overall survival) for a patient having GBM based on machine learning features that describe a histopathology and/or morphology of the one or more digitized images 209. In some embodiments, the prognosis 216 may comprise different levels of risk stratification for a patient having GBM based on overall survival. For example, the prognosis 216 may categorize a patient having GBM into a high-risk category, a medium risk category, or a low-risk category.

In some embodiments, the machine learning pipeline 210 includes a plurality of machine learning stages 210a-210b respectively comprising a machine learning algorithm. For example, the plurality of machine learning stages 210a-210b may comprise a first machine learning stage 210a having a first machine learning algorithm and a second machine learning stage 210b having a second machine learning algorithm. The second machine learning stage 210b is downstream of the first machine learning stage 210a.

In some embodiments, the first machine learning stage 210a is configured to segment the digitized images 209 to identify one or more cellular tumor (CT) regions 212. Because tissue blocks obtained during surgical resections/biopsy are often large and contain non-tumor regions (i.e., necrotic tissue and/or background regions), the identification of the one or more CT regions 212, which may have information relating to GBM within the patient 202, is important to enable analysis of relevant tissue. In some embodiments, the first machine learning stage 210a may comprise a machine learning algorithm that utilizes a convolutional neural network (CNN) (e.g., a ResNet model).

In some embodiments, the second machine learning stage 210b is configured to generate one or more machine learning features 214 that describe a histopathology and/or morphology of the one or more CT regions 212 and to generate a prognosis 216 (e.g., an overall survival) of at least one of the one or more patients 202 based upon the one or more machine learning features 214. In some embodiments, the second machine learning stage 210b may comprise a machine learning algorithm including a CNN (e.g., a ResNet-Cox model) configured to generate one or more machine learning features 214 and a linear regression model configured to generate the prognosis 216 from the one or more machine learning features 214.

It will be appreciated that the disclosed methods and/or block diagrams may be implemented as computer executable instructions, in some embodiments. Thus, in one example, a computer-readable storage device may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

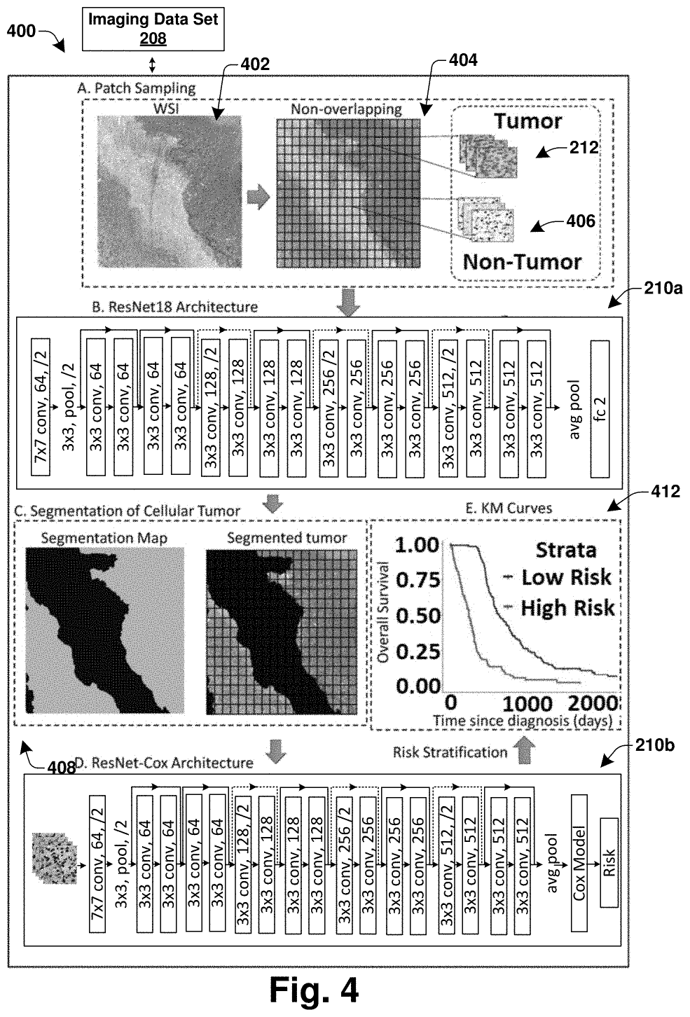

FIG. 3 illustrates some embodiments of a block diagram 300 corresponding to a method and/or apparatus comprising a machine learning pipeline that is configured to determine a prognosis for a patient having GBM.

As shown in block diagram 300, an imaging data set 208 is provided and/or formed. In some embodiments, the imaging data set 208 comprises a plurality of digitized images 209 respectively including digitized biopsy data from surgically resected histopathology slides of GBM. Each of the plurality of digitized images 209 is provided to a machine learning pipeline 210 comprising a plurality of machine learning stages 210a-210b. In some embodiments, the plurality of machine learning stages 210a-210b comprise a first machine learning stage 210a and a second machine learning stage 210b downstream of the first machine learning stage 210a.

The first machine learning stage 210a comprises a first machine learning model (e.g., a first machine learning algorithm) that is configured to identify the one or more CT regions 212 within the plurality of digitized images 209. The first machine learning stage 210a may segment the plurality of digitized images 209 to generate segmented images 304 respectively to have one or more cellular tumor (CT) regions 212 having a high concentration of tumor cells. In some embodiments, the first machine learning stage 210a comprises a first convolutional neural network (CNN) 302 having a plurality of CNN layers 302ₐ-302ₙ (e.g., encoder layers). In some embodiments, the first CNN 302 may comprise a first ResNET model. In some additional embodiments, the first CNN 302 may comprise a ResNet-18 model that has 18 layers. In some embodiments, the segmented images 304 may respectively have one CT region, while in other embodiments the segmented images 304 may respectively comprise multiple CT regions.

The second machine learning stage 210b comprises a second machine learning model (e.g., a second machine learning algorithm) that is configured to identify one or more machine learning features 308 that describe morphologies of the one or more CT regions 212 within the segmented images 304. The second machine learning model is further configured to generate a prognosis 216 based on the one or more machine learning features 308. In some embodiments, the prognosis 216 may be a relative risk of death based an overall survival (OS) of a patient. For example, the prognosis 216 may categorize a patient having a GBM tumor into a high-risk category, a medium-risk category, or a low-risk category based upon the prognosis 216 generated by the machine learning pipeline 210. In some embodiments, the second machine learning stage 210b comprises a second CNN 306 having one or more CNN layers 306ₐ-306ₙ₋₁ and a linear regression model 306n. The one or more CNN layers 306ₐ-306ₙ₋₁ are configured to generate the one or more machine learning features 308, while the linear regression model 306n is configured to determine a prognosis 216 for a patient based upon the one or more machine learning features 308.

In some embodiments, the one or more CT regions 212 may be output from the first CNN 302 as a first matrix. The second CNN 306 is configured to receive the first matrix as an input. The one or more CNN layers 306ₐ-306ₙ₋₁ of the second CNN 306 are configured to operate on the first matrix. In some embodiments, wherein the one or more CNN layers 306ₐ-306ₙ₋₁ act upon one image patch (e.g., a patch obtained from one CT region), the one or more CNN layers 306ₐ-306ₙ₋₁ may generate a vector having the one or more machine learning features (e.g., described as one or more numbers of the vector). In some embodiments, wherein the one or more CNN layers 306ₐ-306ₙ₋₁ act upon a stack of image patches (e.g., an image with multiple CT regions), the one or more CNN layers 306ₐ-306ₙ₋₁ may generate a second matrix with a number of columns that is equal to the number of images patches available in the input stack of the one or more CT regions 212. The one or more machine learning features 308 are provided to the linear regression model 306n, which is configured to generate the prognosis 216 therefrom. The prognosis 216 may comprise one or more risk scores that are indicative of a recurrence of GBM within a patient.

In some embodiments, the second machine learning stage 210b may comprise n-layers. In some embodiments, the linear regression model may be within the final layer (e.g., the n^{th} layer) of the second machine learning stage 210b. In some embodiments, the second machine learning stage 210b may comprise a ResNet model having one or more ResNet layers and one or more layers comprising a linear regression 306n model. For example, in some embodiments the second machine learning stage 210b comprises a ResNet-18 model (e.g., a CNN with 18 layers) that has a final layer (e.g., an 18^{th} layer) replaced with a Cox proportional-hazards model (i.e., a Cox layer), resulting in the second machine learning stage 210b having a ResNet-Cox model with 17 ResNet layers and 1 Cox layer. In some embodiments, the second machine learning stage 210b determines the prognosis 216 from only the or more machine learning features 308, while in other embodiments the second machine learning stage 210b may determine the prognosis based on one or more machine learning features 308 and additional information.

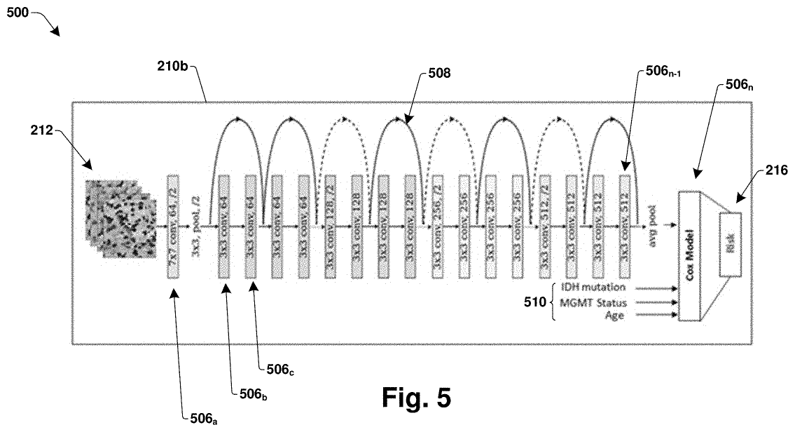

FIG. 4 illustrates some additional embodiments of a block diagram corresponding to a method and/or apparatus comprising a machine learning pipeline that is configured to determine a prognosis for a patient having GBM.

As shown in block diagram 400, an imaging data set 208 is provided and/or formed to include a plurality of digitized images 209 respectively comprising digitized biopsy data from surgically resected histopathology slides of GBM. Each of the plurality of digitized images 209 is provided to a machine learning pipeline 210 comprising a plurality of machine learning stages 210a-210b. In some embodiments, the plurality of machine learning stages 210a-210b may comprise a first machine learning stage 210a and a second machine learning stage 210b downstream of the first machine learning stage 210a.

In some embodiments, to overcome computational challenges with analyzing digitized images 402 having a large size (e.g., digital H&E-stained histopathology WSI containing giga-pixels), non-overlapping patches 404 of a digitized image may be oversampled. In some embodiments, the non-overlapping patches 404 may have a size of approximately 250 pixels×250 pixels. In some embodiments, the non-overlapping patches 404 may be sampled with a stride shift of 0.5× a patch size (e.g., 125 pixels). In some embodiments, non-overlapping patches 404 that contain less than approximately 50% of viable tissue may be discarded. The non-overlapping patches 404 may be sampled from tumor and background/non-tumor regions of a digitized image.

The non-overlapping patches 404 are provided to the first machine learning stage 210a, which is configured to segment the plurality of digitized images 209 to identify CT regions 212. In some embodiments, the non-overlapping patches 404 are used to train the first machine learning stage 210a (e.g., a ResNet-18 model) to classify the non-overlapping patches 404 as belonging to the CT regions 212 and the non-tumor regions 406. In some embodiments, the plurality of non-overlapping patches 404 may be labeled based on majority voting across the pixels of non-overlapping patches 404, with patches from majority tumor regions labeled as 1 and patches from majority background/non-tumor regions labeled as 0. From the CT regions 212 and the non-tumor regions 406, the first machine learning stage 210a may generate segmentation maps 408 of the CT regions 212. In some embodiments, the segmentation maps 408 may comprise patches that have been identified as CT regions 212 while excluding patches that have been identified as non-tumor regions 406.

In some embodiments, the plurality of non-overlapping patches 404 may be separated into CT regions 212 and non-tumor regions 406 using manual segmentation prior to the first machine learning stage 210a. In such embodiments, the manual identification of the CT regions 212 and non-tumor regions 406 may be used to help in training the first machine learning stage 210a to classify each of the non-overlapping patches 404 to either belong to the CT regions 212 or the non-tumor regions 406. In some embodiments, first machine learning stage 210a may be initialized using the pre-trained ImageNet weights and the model training may be performed by minimizing cross-entropy loss. In some embodiments, optimization of the first machine learning stage 210a may be performed using stochastic gradient descent method with an initial learning rate of 0.001. The first machine learning stage 210a may be trained for 80 epochs and the best model may be locked based on the minimum validation loss.

In some embodiments, one or more first augmentation operations may be performed on the plurality of non-overlapping patches 404, prior to being operated upon by the first machine learning stage 210a, to avoid model overfitting. In some embodiments, the one or more first augmentation operations may comprise horizontal and vertical flips, rescaling, rotations, and/or blur augmentation. In some embodiments, one or more second augmentation operations may be performed on the plurality of non-overlapping patches 404, prior to being operated upon by the first machine learning stage 210a, to mitigate stain variability across slides (e.g., obtained from different sites). In some embodiments, the one or more second augmentation operations may comprise randomly perturbing the hue, saturation, contrast, and/or brightness of each patch. In some embodiments, at most 1000 patches may be separately extracted from a viable tumor and non-tumor region of a digitized images.

In some embodiments, following identification of CT regions 212 one WSI per patient may be provided to the second machine learning stage 210b. The WSI is selected to contain a maximum tumor area to slide area. In other embodiments, one or more patches identified as the CT regions 212 may be provided to the second machine learning stage 210b on a patch-by-patch basis. In such embodiments, the second machine learning stage 210b will operate on the patches individually. In some embodiments, after the first machine learning stage 210a is completed, patches identified as the CT regions 212 may be broken into additional non-overlapping patches 410 that are used to train the second machine learning stage 210b. In some embodiments, the non-overlapping patches 404 and the additional non-overlapping patches 410 may have a same size (e.g., 250 pixels×250 pixels), while in other embodiments the non-overlapping patches 404 and the additional non-overlapping patches 410 may have different sizes.

In some embodiments, the second machine learning stage 210b is configured to operate on the patches identified as the CT regions 212 to generate machine learning features that can be used to predict a hazard ratio of a patient. In some embodiments, the hazard ratio may be denoted as a risk score that numerically summarizes an overall survival of the patient. The risk score provides an estimate of relative risk, such that a lower risk score means the patient is more likely to survive. In some embodiments, the second machine learning stage 210b may be trained using a negative partial log likelihood. In some embodiments, to ensure that the second machine learning stage 210b is able to efficiently determine an overall survival of a patient, pre-trained weights of a ResNet18 model used in the first machine learning stage 210a may be transferred to CNN layers of the second machine learning stage 210b. In some embodiments, the second machine learning stage 210b may comprise a ResNet-Cox model having encoder layers initialized with the pretrained ResNet18 encoder weights, and a Cox layer that is randomly initialized. In some such embodiments, the ResNet-Cox model may be subsequently finetuned using OS and/or censor information for 100 epochs using batch size of 32.

In some embodiments, the risk score may be determined on a patch-by-patch basis, so that a CT region 212 comprises a plurality of patches respectively having different risk scores. In some embodiments, the risk score may comprise a patient-level risk scores that is a median of risk scores associated with patches over an entirety of the segmentation map 408 of each patient. In some embodiments, the machine learning pipeline 210 may utilize the patient-level risk scores to generate Kaplan-Meier (KM) curves 412. In some embodiments, low-risk and high-risk groups may be stratified based on a median of risk scores after performing cross-validation on a training set. The median risk score may be then used as a threshold to stratify the patients as belonging to “high-risk” and “low-risk”.

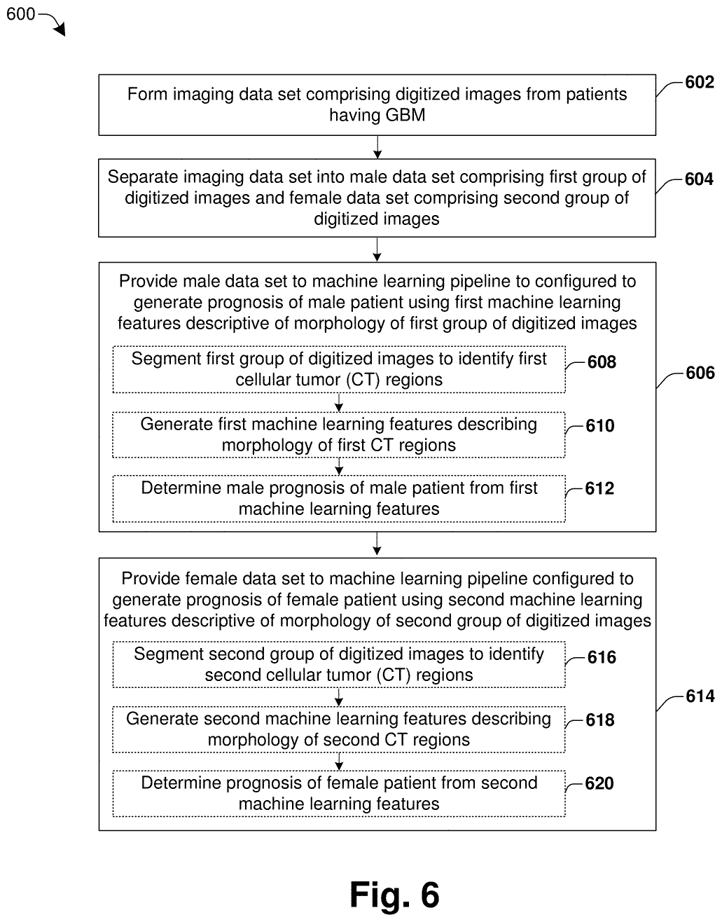

FIG. 5 illustrates a block diagram 500 showing some embodiments of a second machine learning stage within a disclosed machine learning pipeline.

As shown in block diagram 500, the second machine learning stage 210b comprises an architecture of a ResNet-Cox model that contains encoder layers of a ResNet-18 model. The last layer of the ResNet-18 model is replaced with a Cox proportional-hazards model. Therefore, the second machine learning stage 210b comprises 17 ResNET layers 506ₐ-506ₙ₋₁ followed by one layer including a Cox proportional-hazards model 506ₙ. In some embodiments, the ResNet layers 506ₐ-506ₙ₋₁ may comprise convolutions that are 3×3, while in other embodiments the ResNet layers 506ₐ-506ₙ₋₁ may comprise convolutions having different size (e.g., 5×5, 7×7, etc.).

In some embodiments, the output of the final ResNet layer 506ₙ₋₁ is provided to the Cox proportional-hazards model 506ₙ. Based on the output of the final ResNet layer 506ₙ₋₁, the Cox proportional-hazards model 506ₙ may generate a prognosis 216 (e.g., an overall survival denoted as a risk score). In some embodiments, the ResNet layers 506ₐ-506ₙ₋₁ may comprise skip connections 508 that are configured to add an output from an earlier layer to a later layer. The skip connections 508 are configured to mitigate the vanishing gradient problem and improve an accuracy of the second machine learning stage 210b.

In some embodiments, in addition to the output of the final ResNet layer 506ₙ₋₁, the Cox proportional-hazards model 506ₙ may also receive one or more multi-modal inputs 510 from which to base the prognosis 216. The one or more multi-modal inputs 510 allow for the second machine learning stage 210b to generate a multi-modal ResNet-Cox model that takes into account additional inputs when determining the prognosis 216 (e.g., overall survival). In some embodiments, the one or more multi-modal inputs 510 may comprise transcriptomic data, IDH mutations, O(6)-Methylguanine-DNA-methyltransferase (MGMT) status, an age of a patient, or the like.

It has been appreciated that gender is an important factor that may impacts prognosis, drug response, and/or survival outcomes in GBM patients. For example, sexual-dimorphism in GBM points to a worse overall survival (OS) in males versus females, independent of extent of resection, treatment, and/or age. Furthermore, increased tumorigenesis and higher proliferation rates have been observed in male specific GBM astrocytes compared to female specific GBM astrocytes, suggesting a potentially unfavorable response to conventional treatments in GBM male patients. Therefore, sexual-dimorphism in GBM may provide for differences in patients that could guide personalized treatment decisions.

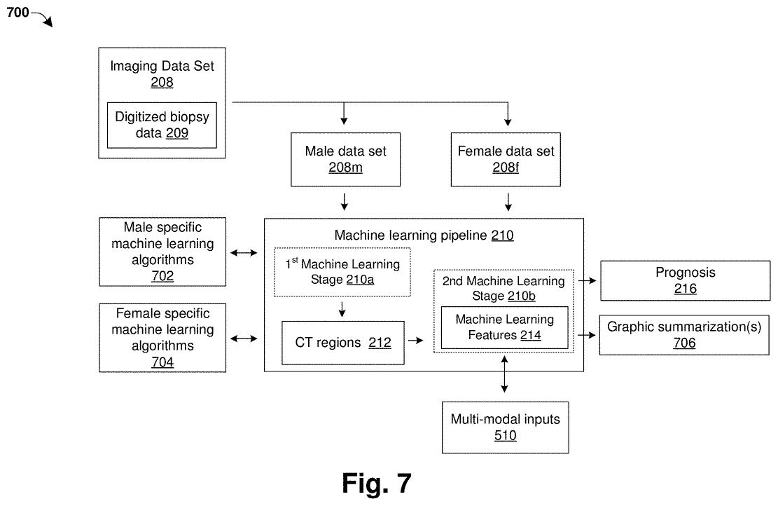

Accordingly, in some embodiments the disclosed machine learning pipeline (e.g., deep learning pipeline) may be configured to utilize gender specific machine learning models (e.g., machine learning algorithms) to determine a prognosis of a patient. In some additional embodiments, the disclosed machine learning pipeline may leverage additional multi-modal inputs (e.g., transcriptomic data) to extend the “sex-specific” prognostic models to provide a cross-scale molecular understanding of differences across males and female GBM patients, and build comprehensive and patient-centric treatment plans. FIG. 6 illustrates a flow diagram of some embodiments of a method of utilizing a gender specific machine learning pipeline to determine a prognosis of a patient having GBM.

At act 602, an imaging data set comprising one or more digitized images is provided and/or formed for patients having GBM.

At act 604, the imaging data set is separated into a male data set comprising a first group of digitized images that are predominantly (e.g., exclusively) from male patients and a female data set comprising a second group of digitized images that are predominantly (e.g., exclusively) from female patients.

At act 606, the male data set is provided to a machine learning pipeline having first machine learning algorithms (e.g., first deep learning algorithms) configured to generate a male prognosis (e.g., a male overall survival) of a male patient using first machine learning features describing a morphology of the first group of digitized images. In some embodiments, the first machine learning algorithms may generate a prognosis according to acts 608-612.

At act 608, the first group of digitized images are segmented to identify one or more first CT regions within the first group of digitized images. In some embodiments, the segmentation may separate the first CT regions from background and/or necrotic regions.

At act 610, first machine learning features are generated for the first CT regions. The first machine learning features may describe a morphology of the first CT regions.

At act 612, a male prognosis of the male patient is determined from the first machine learning features.

At act 614, the female data set is provided to a machine learning pipeline having second machine learning algorithms (e.g., second deep learning algorithms) configured to generate a female prognosis (e.g., a female overall survival) of a female patient using second machine learning features describing a morphology of the second group of digitized images. In some embodiments, the second machine learning algorithms may generate a prognosis according to acts 616-620.

At act 616, the second group of digitized images are segmented to identify one or more second CT regions within the second group of digitized images. In some embodiments, the segmentation may separate the second CT regions from background and/or necrotic regions.

At act 618, second machine learning features are extracted from the second CT regions. The second machine learning features may describe a morphology of the second CT regions.

At act 620, a female prognosis of the female patient is determined from the second machine learning features.

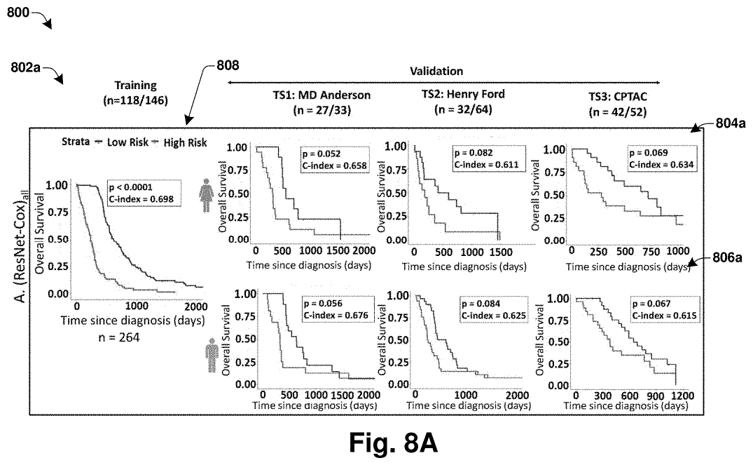

FIG. 7 illustrates some embodiments of a block diagram 700 corresponding to a method and/or apparatus comprising a machine learning pipeline that is configured to determine a prognosis for a patient having GBM.

As shown in block diagram 700, an imaging data set 208 is provided and/or formed to include a plurality of digitized images 209. The plurality of digitized images 209 within the imaging data set 208 are divided into a male data set 208m and a female data set 208f based on a biological gender (e.g., male or female) of a patient associated with a digitized image. The male data set 208m comprises a first group of digitized images that are predominantly (e.g., exclusively) from male patients. The female data set 208f comprises a second group of digitized images that are predominantly (e.g., exclusively) from female patients.

Digitized images within the male data set 208m and the female data set 208f are separately provided to a machine learning pipeline 210 comprising a plurality of machine learning stages 210a-210b. The machine learning pipeline 210 may comprise a first machine learning stage 210a and a second machine learning stage 210b downstream of the first machine learning stage 210a. The machine learning pipeline 210 utilizes machine learning algorithms specifically corresponding to males and females to separately acts on the digitized images within the male data set 208m and the female data set 208f. For example, the machine learning pipeline 210 may act on the digitized images within the male data set 208m using male specific machine learning algorithms 702 (i.e., (ResNet-Cox)_{M}) that correspond to male patients, while the machine learning pipeline 210 may separately act on the digitized images within the female data set 208f using female specific machine learning algorithms 704 (i.e., (ResNet-Cox)_{F}) that correspond to female patients.

Based on the male specific machine learning algorithms 702 and/or the female specific machine learning algorithms 704, the machine learning pipeline 210 will generate separate prognosis for male and female patients. Because the male specific machine learning algorithm 702 and/or the female specific machine learning algorithm 704 take into account the sexual-dimorphism in GBM, they are able to provide for differences in patient outcomes that may guide personalized treatment decisions.

In some embodiments, a second machine learning stage 210b of the machine learning pipeline 210 may be configured to utilize one or more multi-modal inputs 510 to generate the prognosis 216. In some such embodiments, a Cox proportional-hazards model of the second machine learning stage 210b may be configured to receive the one or more multi-modal inputs 510 in addition to the output of one or more CNN layers of the second machine learning stage 210b. In some embodiments, the one or more multi-modal inputs 510 may include clinical and molecular information of patients, such as IDH mutations, O(6)-Methylguanine-DNA-methyltransferase (MGMT) status, an age of a patient, or the like. In some embodiments, the one or more multi-modal inputs 510 may be used by gender specific multi-modal machine learning algorithms. For example, the one or more multi-modal inputs 510 may be used by male multi-modal machine learning algorithms (e.g., including a multi-modal male ResNet-Cox model) and female multi-modal machine learning algorithms (e.g., including a multi-modal female ResNet-Cox model).

In some embodiments, the machine learning pipeline 210 may be further configured to generate one or more graphic summarizations 706 corresponding to the prognosis 216. The one or more graphic summarizations 706 may be used (e.g., by pathologists) to correlate model predictions with underlying pathophysiology of GBM so as to allow for health care professionals to better understand the causes and mechanisms of the GBM.

In some embodiments, the machine learning pipeline 210 may employ t-distributed stochastic neighbor embedding (t-SNE) to generate one or more graphic summarizations 706 comprising 2D t-SNE plots. The t-SNE plots may be used to identify the key morphological niches that may be linked to improved and poor prognosis in GBM, and may correlate with expert pathologist interpretations

In some embodiments, the machine learning pipeline 210 may be configured to generate one or more graphic summarizations 706 comprising one or more risk density maps. The risk density maps highlight regions within a tumor-microenvironment that contribute to survival risk-prediction (e.g., to highlight specific niches in a tumor microenvironment that contributed to the risk prediction). In some embodiments, the risk density maps may be generated using a predicted risk for every patch in a WSI.

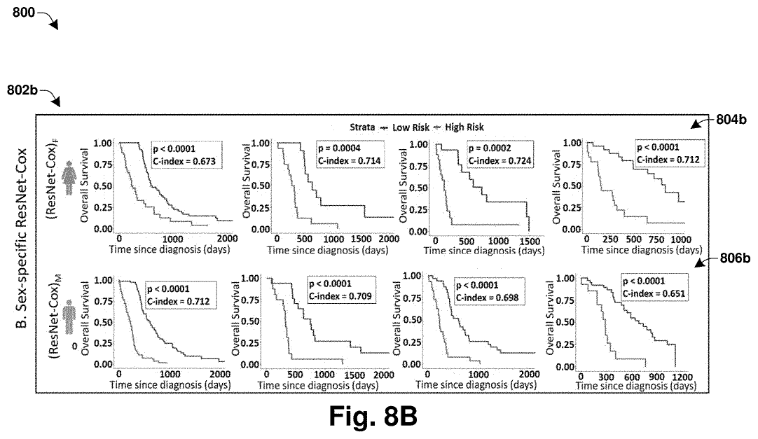

FIG. 8 illustrates some exemplary Kaplan-Meier (KM) survival curves 800 generated by a disclosed machine learning pipeline.

The KM survival curves 800 are shown for three different classes of machine learning pipelines 802a-802c (e.g., deep learning pipelines). The three different classes of machine learning pipelines 802a-802c comprise a first class of machine learning pipelines 802a that utilize gender neutral machine learning algorithms that do not take into consideration gender, a second class of machine learning pipelines 802b that utilize gender specific machine learning algorithms that take into consideration gender, and a third class of machine learning pipelines 802c that utilize gender specific multi-modal machine learning algorithms that take into consideration gender as well as multi-modal inputs. The KM survival curves 800 for each of the different classes of machine learning pipelines 802a-802c are shown for female groups 804a-804c and for male groups 806a-806c across training and three test sets (i.e., validation sets). For the first class of machine learning pipeline 802a, a KM survival curve is also shown for a gender neutral group 808.

For each of the KM survival curves 800, the x-axis represents an overall survival (OS) in days and y-axis represents the estimated survival probability. The KM survival curves 800 for the three different classes of machine learning pipelines 802a-802c, respectively have a high risk and a low-risk stratification. The high risk and the low-risk stratification is based on a median of the risk scores generated by the disclosed machine learning pipeline.

The first class of machine learning pipeline 802a does not show significant differences (p>0.05) in the KM survival curves of male groups 804a and female groups 804b across the test sets. In the second class of machine learning pipeline 802b, concordance indices (C-indices) for the training set and the three test sets are (0.673, 0.714, 0.724, 0.712) and p-values are (<0.0001, 0.0004, 0.0002, <0.0001), respectively using a female specific machine learning pipeline for female groups 806a, while the p-value is <0.0001 and the C-indices are (0.712, 0.709, 0.698, 0.651) using a male specific machine learning pipeline for male groups 806b. In the third class of machine learning pipeline 802c, C-indices for the training and the three test sets are (0.696, 0.736, 0.731, 0.729) and p-values are (<0.0001, <0.0001, 0.0002, <0.0001) using a female specific multi-modal machine learning pipeline for female groups 804c, while p is <0.0001 and the C-indices are (0.729, 0.738, 0.724, 0.696) using a male specific multi-modal machine learning pipeline for male groups 806c.

Therefore, as illustrated by the KM survival curves 800 of FIG. 8, the gender specific multi-modal machine learning pipelines, which comprise paired genomic and histologic information along with patients' age, have improved C-indices and shows statistically significant differences across patients at low versus high-risk across both the training set as well as the test sets. This suggest that the patients' age, IDH, and MGMT status could complement the prognostic ability of the histologic risk score in predicting OS.

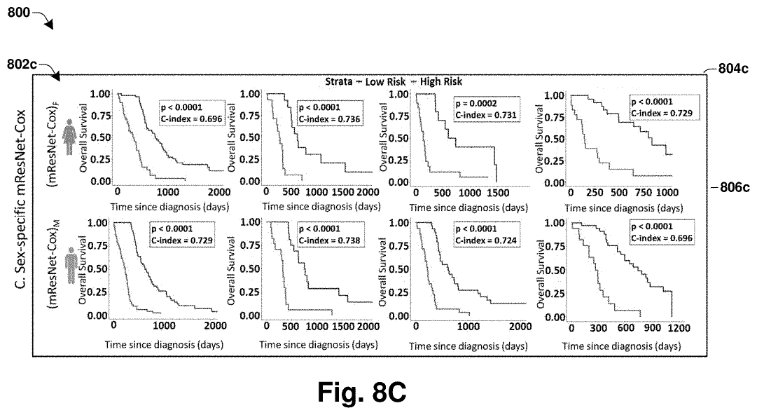

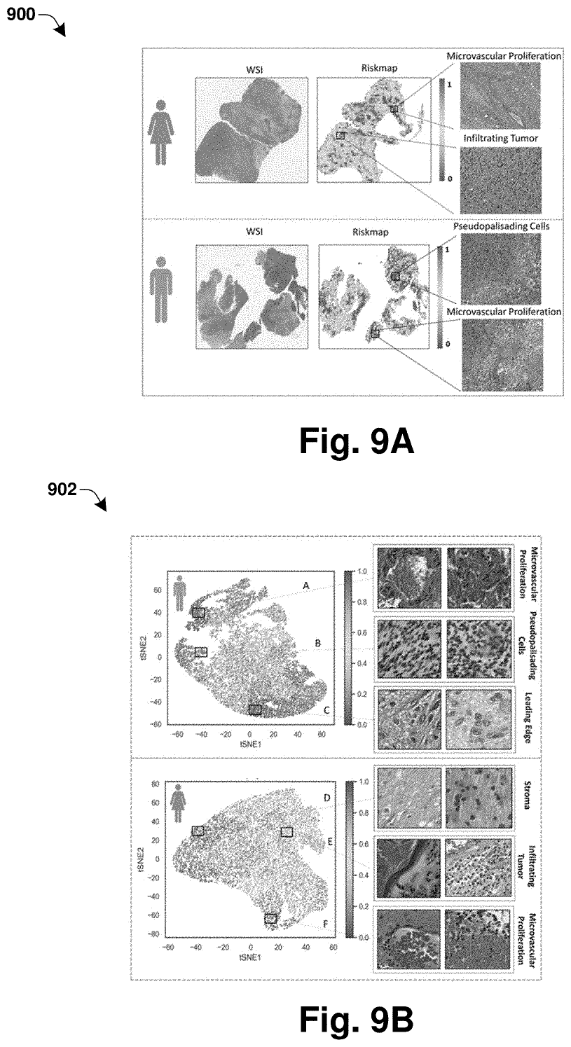

FIG. 9A illustrates some exemplary risk density maps 900 generated by a disclosed machine learning pipeline.

In some embodiments, the risk density maps 900 may be generated using a predicted risk for every patch in a WSI generated using gender specific machine learning pipelines. In some embodiments, the predicted risks obtained across patches may be aggregated to WSI-level. A color map representing risk may be overlaid on every WSI, such that red and blue indicated high and low-risk regions, respectively. In some embodiments, activation maps (i.e., 512 dimensional features) may be extracted for each patch before feeding them to the final fully connected Cox-layer of the second stage of the male specific deep learning model ((ResNet-Cox)_{M}) and the second stage of the female specific deep learning model ((ResNet-Cox)_{F}).

FIG. 9B illustrates some exemplary t-SNE (t-distributed Stochastic Neighbor Embedding) plots 902 generated by a disclosed machine learning pipeline.

In the t-SNE plots 902, each point represents a patch and is colored as per the predicted risk-score using the gender specific machine learning pipelines. The red cluster represents patches with relatively higher-risk score compared to the blue cluster. A few representative patches are shown in A-F. In some embodiments, the patches may be generally clustered by their predicted risk-scores.

In some embodiments, the t-SNE plots 902 may be formed by randomly sampling patches from tumor regions of the WSI from test sets. For example, 100 patches may be randomly sampled from the tumor regions of each WSI from the test sets independently. Following sampling, a total of 10,100 patches from male data sets and 14,900 patches from female data sets may be obtained and then reduced from a 512-dimensional feature set to a two-dimensional (2D) space via t-SNE. Each dot on the 2D t-SNE plot represents a sampled tile and is color-coded according to an associated patch-level risk score obtained using (ResNet-Cox)_{M} and (ResNet-Cox)_{F} survival models.

As shown in both the risk density maps 900 of FIG. 9A and the t-SNE plots 902 of FIG. 9B, the patches that belong to higher risk scores in males correspond to endothelial cell hypertrophy (i.e., microvascular proliferation (MVP)) and pseudopalisading cells, while low-risk patches in males belong to peritumoral regions (i.e., leading edge). MVP represents endothelial cell hypertrophy that stimulates new blood-vessel formation and augments vascular permeability. Pseudopalisading cells create hypoxic niche for cancer stem cells (CSCs) that results in increased secretion of hypoxia inducible factor (HIF). Hypoxia protects tumor cells and CSCs from chemotherapy and radiotherapy. This may be a cause of tumor progression, poor survival, and treatment resistance in male GBMs. Additionally, HIF promotes neovascularization and recruit innate immune cells including tumor-associated macrophages (TAMs) that are often considered to be facilitators of tumor growth because of their proangiogenic and immunosuppressive properties. This suggests that high-risk male patients may be better candidates for immunotherapy to stimulate their antitumor immunity

Similarly, in females patches with high-risks belong to infiltrating tumor and MVP, while the low-risk patches correspond to regions with stroma. The proliferative (MVP) nature of GBM tumor augments vascular permeability, stimulates new blood-vessel formation, and is responsible for extensive tumor progression. Infiltrating and proliferative endothelial cells (MVP) promote angiogenesis and regulated by various integrins. Thus, drugs targeting integrin signaling may be more effective for high-risk females as integrins are used for cell invasion, migration, and tumor progression.

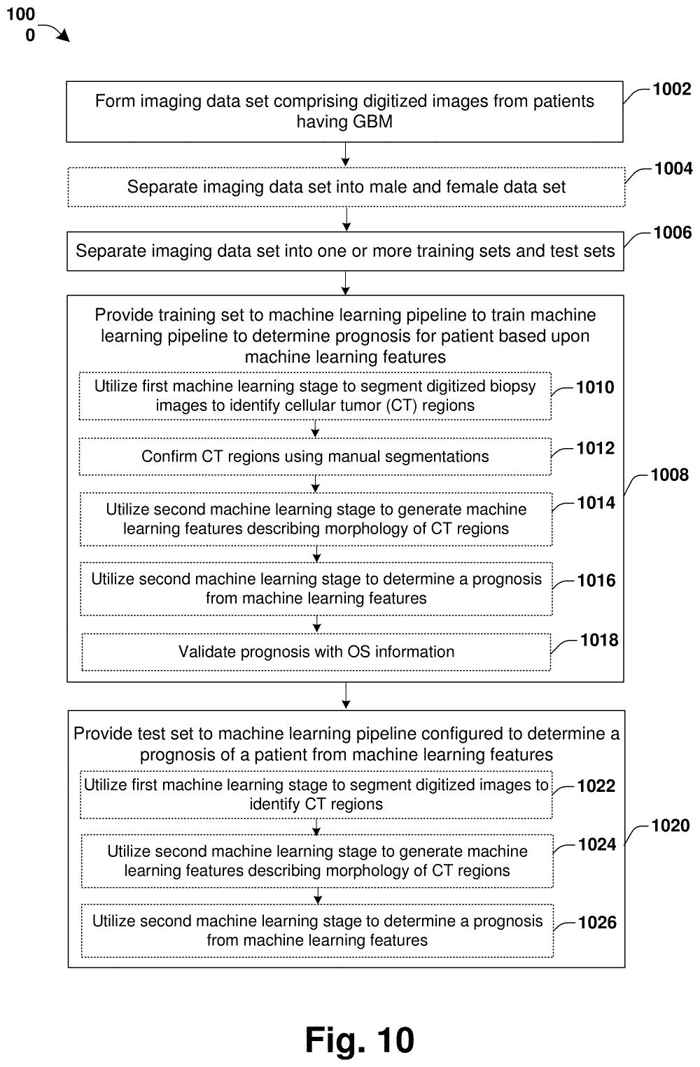

FIG. 10 illustrates some embodiments of a method 1000 of generating a machine learning pipeline to determine a prognosis of a patient having GBM with training and test sets.

At act 1002, an imaging data set comprising digitized images is provided and/or formed for a plurality of patients having GBM.

At act 1004, the imaging data set may be separated into a male data set predominantly (e.g., exclusively) comprising a first group of digitized images from male patients and a female data set predominantly (e.g., exclusively) comprising a second group of digitized images from female patients.

At act 1006, the imaging data set is separated into one or more training sets and one or more test sets. The one or more training sets respectively comprise a first plurality of digitized images (from the plurality of digitized images) and the test set respectively comprise a second plurality of digitized images (from the plurality of digitized images). In some embodiments, the male data set may be separated into a male training set and a male test set while the female data set may be separated into a female training set and a female test set. In some embodiments, the imaging data set may be separated into the one or more training sets and the one or more test sets on a patch-by-patch basis. For example, the plurality of digitized images may be separated into patches, the patches may be identified as tumor or non-tumor regions, and the patches identified as tumor regions may be separated into the one or more training sets and the one or more tests sets. In some embodiments, 80% of the patches identified as tumor regions may be placed in the one or more training sets while the remaining 20% of patches identified as non-tumor regions may be placed in the one or more test sets.

At act 1008, the first plurality of digitized images within one of the one or more training sets are provided to a machine learning pipeline to train the machine learning pipeline to determine a prognosis (e.g., an overall survival) of a patient based on machine learning features. In some embodiments, the trained machine learning algorithms may be generated according to acts 1010-1018.

At act 1010, a first machine learning stage is used to segment the first plurality of digitized images. Segmenting the digitized images identifies cellular tumor (CT) regions within the first plurality of digitized images.

At act 1012, the CT regions may be confirmed using manual segmentations having CT annotations that were confirmed by expert neuropathologists. In some embodiments, the first machine learning stage may be adjusted in response to the confirmation.

At act 1014, a second machine learning stage is used to generate one or more machine learning features that describe a morphology of the CT regions. In some embodiments, the second machine learning stage may comprise one or more CNN layers (e.g., ResNet layers) configured to generate the one or more machine learning features.

At act 1016, the second machine learning stage is used to determine a prognosis (e.g., an overall survival) of a patient from the one or more machine learning features. In some embodiments, the second machine learning stage may comprise a linear regression layer (e.g., a Cox proportional-hazards model) configured to determine the prognosis.

At act 1018, the prognosis is validated using overall survival information related to patients of the digitized images.

It will be appreciated that acts 1010-1018 may be repeated multiple times during training of the machine learning pipeline to generate the trained machine learning algorithms including a trained first machine learning algorithm and/or the trained second machine learning algorithm. It will also be appreciated that acts 1010-1018 may be performed one or more times for each of the one or more training and test sets. For example, acts 1010-1018 may be performed a first plurality of times on a first male training set to generate first trained machine learning algorithms that correspond to male patients and a second plurality of times on a first female training set to generate second trained machine learning algorithms that correspond to female patients.

At act 1020, the second plurality of digitized images within one of the one or more test sets are provided to the machine learning pipeline to determine a prognosis of a patient. The machine learning pipeline is configured to use the trained machine learning algorithms to determine the prognosis. In some embodiments, the machine learning pipeline may determine the prognosis of the patient according to acts 1022-1026.

At act 1022, the first machine learning stage is used to segment the second plurality of digitized images. In some embodiments, the first machine learning stage may comprise one or more CNN layers (e.g., ResNet layers) configured to utilize the trained machine learning algorithms to segment the second plurality of digitized images.

At act 1024, the second machine stage is used to generate one or more machine learning features that describe a morphology of the CT regions. In some embodiments, the second machine learning stage may comprise one or more CNN layers (e.g., ResNET layers) configured to utilize the trained machine learning algorithms to generate the one or more machine learning features.

At act 1026, the second machine learning stage is used to determine a prognosis (e.g., an overall survival) of a patient from the one or more machine learning features. In some embodiments, the second machine learning stage may comprise a linear regression layer (e.g., a Cox proportional-hazards model) configured to utilize the trained machine learning algorithms to determine the prognosis.

It will be appreciated that acts 1022-1026 may be repeated multiple times during testing of the machine learning pipeline. It will also be appreciated that acts 1022-1026 may be performed one or more times for each of the one or more training and test sets. For example, acts 1022-1026 may be performed a first plurality of times on a first male training set and a second plurality of times on a first female training set.

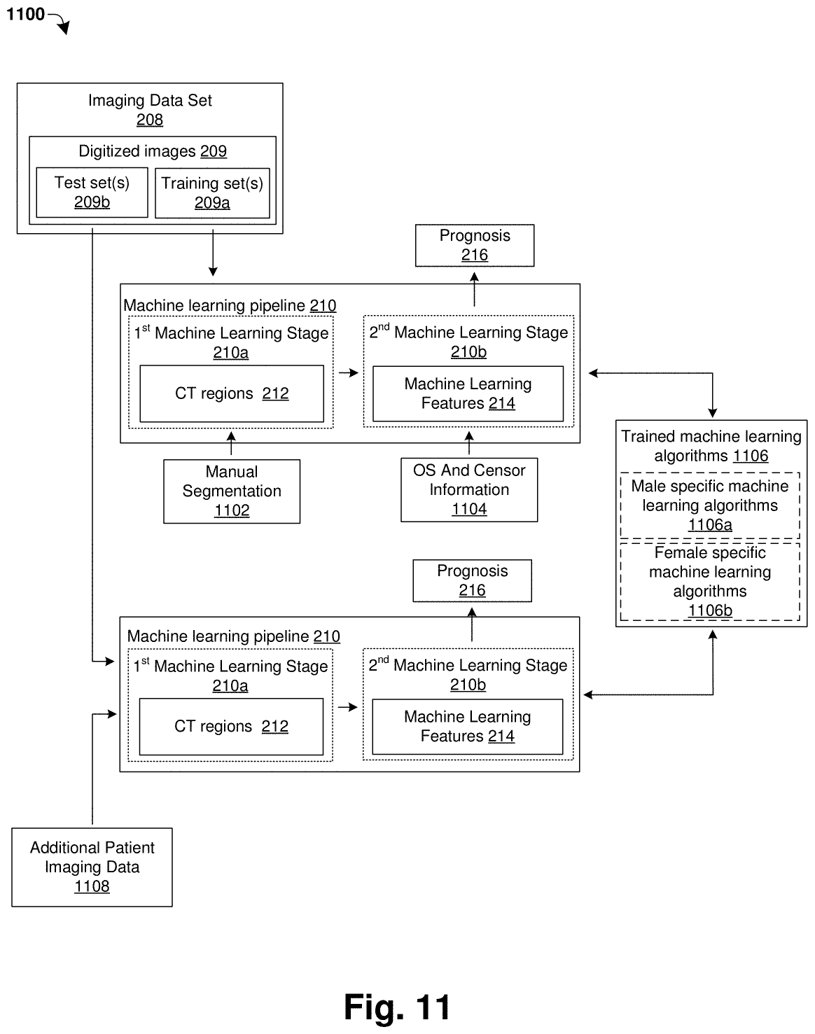

FIG. 11 illustrates some embodiments of a block diagram 1100 corresponding to a method and/or apparatus comprising a machine learning pipeline that is configured to determine a prognosis for a patient having GBM.

As shown in block diagram 1100, an imaging data set 208 is provided and/or formed to comprise a plurality of digitized images 209. In some embodiments, the imaging data set 208 is divided into one or more training sets 209a and one or more test sets 209b. In some embodiments, the one or more training sets 209a may comprise one or more male training sets and one or more female training sets. In some embodiments, the one or more test sets 209b may comprise one or more male test sets and one or more female test sets.

The one or more training sets 209a are provided to a machine learning pipeline 210 having a first machine learning stage 210a and a second machine learning stage 210b. The machine learning pipeline 210 is configured to utilize the one or more training sets 209a to generate trained machine learning algorithms 1106. In some embodiments, the trained machine learning algorithms 1106 may comprise male specific machine learning algorithms 1106a and female specific machine learning algorithms 1106b. In some embodiments, the first machine learning stage 210a is configured to operate on the one or more training sets 209a in a manner that segments the digitized images within the one or more training sets 209a to identify CT regions 212. The CT regions 212 are then provided to the second machine learning stage 210b. The second machine learning stage 210b is configured to operate on the CT regions 212 to generate machine learning features 214 that describe a morphology of the one or more training sets 209a. The second machine learning stage 210b is further configured to utilize the machine learning features 214 to generate a prognosis 216.

In some embodiments, manual segmentations 1102 may be provided to the first machine learning stage 210a to improve an accuracy of the first machine learning stage 210a. In some embodiments, overall survival and/or censor information 1104 may be provided to the second machine learning stage 210b to improve an accuracy of the second machine learning stage 210b.

The one or more test sets 209b are provided to the machine learning pipeline 210. The machine learning pipeline 210 is configured to utilize the trained machine learning algorithms 1106 to operate on the one or more test sets 209b in a manner that segments the digitized images within the one or more test sets 209b to identify CT regions 212. The CT regions 212 are then provided to the second machine learning stage 210b. The second machine learning stage 210b is configured to operate on the CT regions 212 to generate machine learning features 214 that describe a morphology the one or more test sets 209b. The second machine learning stage 210b is further configured to utilize the machine learning features 214 to generate a prognosis 216.

In some embodiments, one or more additional digitized images 1108 from an additional patient may be provided to the machine learning pipeline 210. The machine learning pipeline 210 is configured to receive the additional digitized image 1108 and to generate a prognosis 216 for the additional patient utilizing the trained machine learning algorithms 1106.

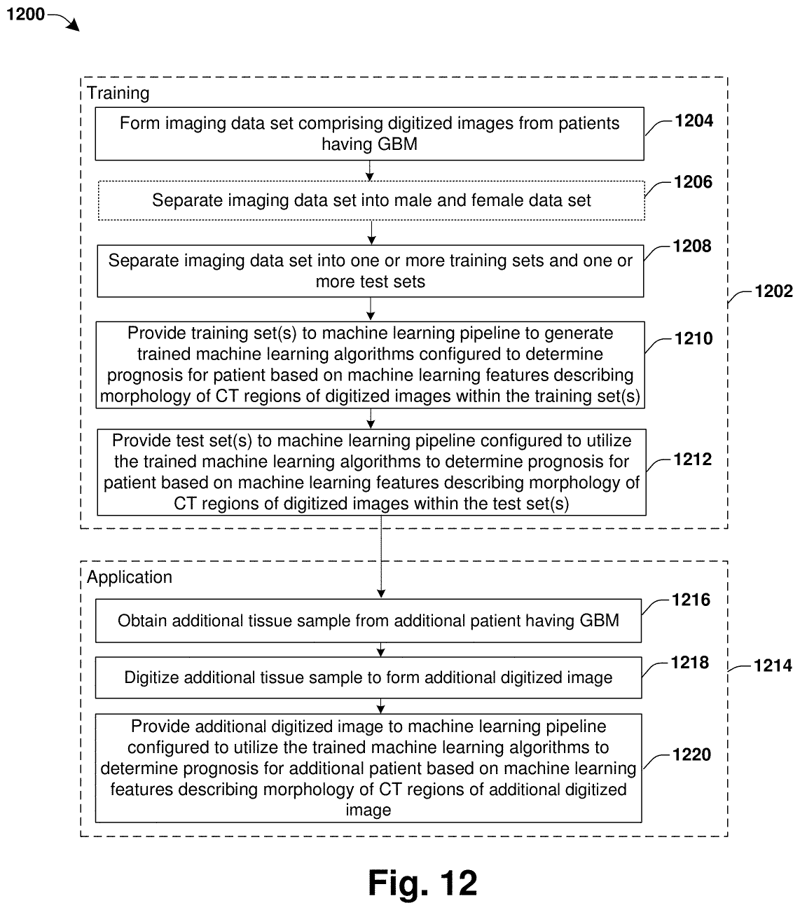

FIG. 12 illustrates some additional embodiments of a method of generating a machine learning pipeline that is configured to determine a prognosis of a patient having GBM and applying the machine learning pipeline to an additional patient.

The method 1200 comprises a training phase 1202 and an application phase 1214. The training phase 1202 is configured to generate a machine learning pipeline that is able to provide a prognosis (e.g., an overall survival) of a patient using one or more morphological features of digitized biopsy images from surgically resected GBM. In some embodiments, the training phase 1202 may be performed according to acts 1204-1212.

At act 1204, an imaging data set is provided and/or formed to comprise digitized images from patients having GBM.

At act 1206, the imaging data set may be separated into a male data set and a female data set.

At act 1208, the imaging data set is separated into one or more training sets and one or more test sets. In some embodiments, the male data set may be separated into one or more male training sets and the one or more male test sets and the female data set may be separated into one or more female training sets and one or more female test sets.

At act 1210, the one or more training sets are provided to a machine learning pipeline (e.g., a deep learning pipeline) to generate trained machine learning algorithms. The machine learning pipeline is configured to utilize the trained machine learning algorithms to determine prognosis for a patient from one or more machine learning features that describe a morphology of the CT regions within the one or more training sets.

At act 1212, the one or more test sets are provided to the machine learning pipeline. The machine learning pipeline is configured to utilize the trained machine learning algorithms to determine a prognosis of a patient from one or more machine learning features that describe a morphology of the CT regions within the one or more test sets.

The application phase 1214 is configured to utilize the machine learning pipeline on one or more additional images, which are taken from an additional patient having GBM, to determine a prognosis of the additional patient.

At act 1216, an additional tissue sample is obtained from an additional patient having GBM.

At act 1218, the additional tissue sample it digitized to form additional digitized image.

At act 1220, the additional digitized image is provided to the machine learning pipeline. The machine learning pipeline is configured to utilize the trained machine learning algorithms to determine a prognosis for the additional patient from one or more machine learning features that describe a morphology of CT regions within the additional digitized image.

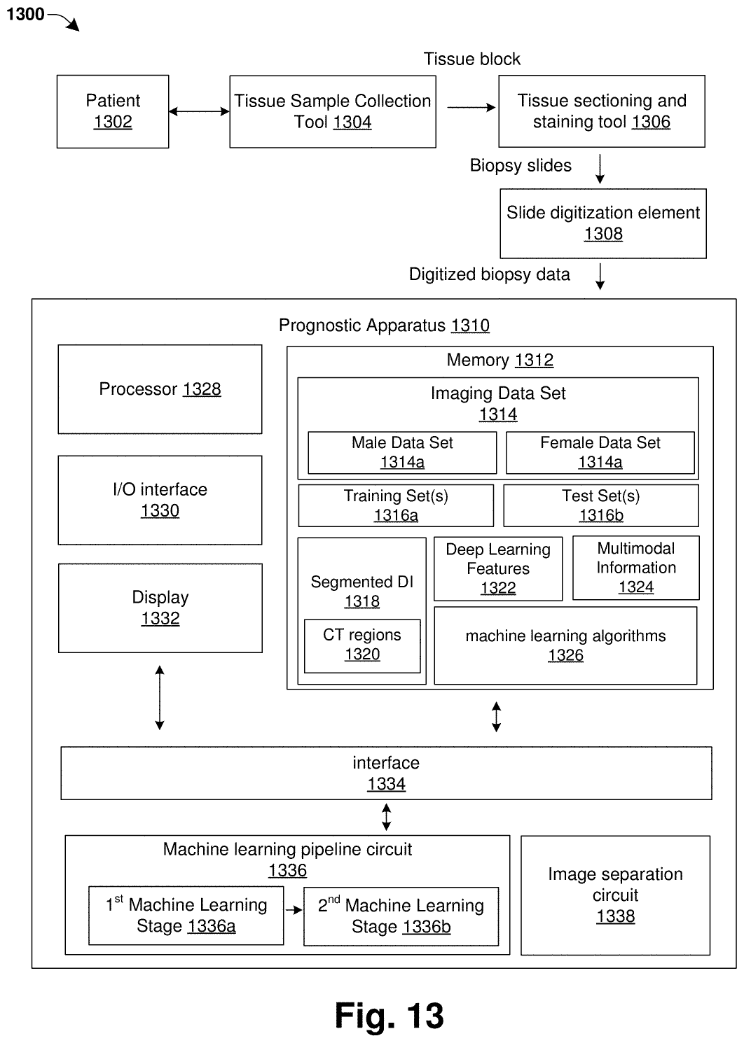

FIG. 13 illustrates some embodiments of an apparatus 1300 comprising a machine learning pipeline that is configured to determine a prognosis of a patient having GBM.

The apparatus 1300 comprises a prognostic apparatus 1310. The prognostic apparatus 1310 is coupled to a slide digitization element 1308 that is configured to obtain digitized images (e.g., whole slide images) of tissue samples collected from a patient 1302 having GBM. In some embodiments, one or more tissue samples (e.g., a tissue block) may be obtained using a tissue sample collection tool 1304 (e.g., a cannular, forceps, needle, punch, or the like). The one or more tissue samples may be provided to a tissue sectioning and staining tool 1306. In some embodiments, the tissue sectioning and staining tool 1306 may be configured to slice the one or more tissue samples into thin slices that are placed on transparent slides (e.g., glass slides) to generate biopsy slides. The tissue on the biopsy slides is then stained by applying a dye. The dye may be applied on the posterior and anterior border of the sample tissues to locate the diseased or tumorous cells or other pathological cells. In some embodiments, the biopsy slides may comprise H&E (Hematoxylin and Eosin) stained slides. The slide digitization element 1308 is configured to convert the biopsy slides to digitized biopsy data (e.g., whole slide images). In some embodiments, the slide digitization element 1308 may comprise an image sensor (e.g., a photodiode, CMOS image sensor, or the like) that is configured to capture a digital image of the biopsy slides.

The prognostic apparatus 1310 comprises a processor 1328 and a memory 1312. The processor 1328 can, in various embodiments, comprise circuitry such as, but not limited to, one or more single-core or multi-core processors. The processor 1328 can include any combination of general-purpose processors and dedicated processors (e.g., graphics processors, application processors, etc.). The processor(s) 1328 can be coupled with and/or can comprise memory (e.g., memory 1312) or storage and can be configured to execute instructions stored in the memory 1312 or storage to enable various apparatus, applications, or operating systems to perform operations and/or methods discussed herein.

Memory 1312 can be configured to store an imaging data set 1314 comprising digitized images for a plurality of patients having GBM. The digitized images may comprise digitized biopsy images having a plurality of pixels, each pixel having an associated intensity. In some additional embodiments, the digitized images may be stored in the memory 1312 as one or more training sets 1316a of digitized images for training a classifier and/or one or more test sets 1316b (e.g., validation sets) of digitized images.

The prognostic apparatus 1310 also comprises an input/output (I/O) interface 1330 (e.g., associated with one or more I/O devices), a display 1332, a machine learning pipeline circuit 1336, an image separation circuit 1338, and an interface 1334 that connects the processor 1328, the memory 1312, the I/O interface 1330, the machine learning pipeline circuit 1336, and the image separation circuit 1338. I/O interface 1330 can be configured to transfer data between the memory 1312, the processor 1328, the machine learning pipeline circuit 1336, and external devices, for example, the slide digitization element 1308. The display 1332 is configured to output or display the prognosis the prognostic apparatus 1310.

In some embodiments, the machine learning pipeline circuit 1336 may comprise a first machine learning stage 1336a configured to segment the plurality of digitized images within the imaging data set 1314 to generate segmented images 1318 respectively having one or more CT regions 1320. In some embodiments, the first machine learning stage 1336a comprises a first CNN model (e.g., a first ResNet model having a plurality of ResNet layers).

In some embodiments, the machine learning pipeline circuit 1336 may further comprise a second machine learning stage 1336b downstream of the first machine learning stage 1336a. The second machine learning stage 1336b comprises a plurality of CNN layers (e.g., a plurality of ResNet layers) configured to extract one or more machine learning features 1322 from the one or more CT regions 1320. The second machine learning stage 1336b further comprises a linear regression model (e.g., a cox regression model) that is configured to generate a prognosis from one or more machine learning features 1322. In some embodiments, the machine learning pipeline circuit 1336 may operate according to machine learning algorithms 1326 stored in memory 1312. The machine learning algorithms 1326 may comprise gender neutral algorithms, gender specific algorithms, and/or gender specific multi-modal algorithms, as described above.

In some embodiments, the image separation circuit 1338 may be configured to separate the digitized images based on gender. For example, the image separation circuit 1338 may separate the imaging data set into a male data set 1314a and a female data set 1314b, which are stored in the memory 1312. In some embodiments, the machine learning pipeline circuit 1336 may be configured to operate upon the male data set 1314a and the female data set 1314b separately, so as to separately train a male specific machine learning algorithm and a female specific machine learning algorithm that are configured to respectively generate a prognosis for male and female patients having GBM.

In some embodiments, memory 1312 may be further configured to store multi-modal information 1324 (e.g., IDH mutations, O(6)-Methylguanine-DNA-methyltransferase (MGMT) status, an age of a patient, or the like). The multi-modal information 1324 may be provided to the second machine learning stage 1336b, which may utilize the multi-modal information 1324 to build gender specific multi-modal machine learning algorithms (e.g., gender specific multi-modal ResNet-Cox models).

### Example Use Case 1

The following discussion provides example embodiments in connection with an example use case involving a method of generating a deep learning pipeline that is configured to determine a prognosis of a patient having a GBM tumor.

Purpose: Glioblastoma is an aggressive and universally fatal tumor. Morphological information as captured from cellular regions on surgically resected histopathology slides has the ability to reveal the inherent heterogeneity in Glioblastoma and thus has prognostic implications. In this work, we hypothesized that capturing morphological attributes from high cellularity regions on Hematoxylin and Eosin (H&E)-stained digitized tissue slides using an end-to-end deep-learning pipeline will enable risk-stratification of GBM tumors based on overall survival.

Methods: A large multi-cohort study consisting of N=514 H&E-stained digitized tissue slides along with overall-survival data (OS) was obtained from the Ivy Glioblastoma atlas project (Ivy-GAP (N=41)), TCGA (N=379), and CPTAC (N=94). Our deep-learning pipeline consisted of two stages. First stage involved segmenting cellular tumor (CT) from necrotic-regions and background using Resnet-18 model, while the second stage involved predicting OS, using only the segmented CT regions identified in the first stage. For the segmentation stage, we leveraged the Ivy-GAP cohort, where CT annotations confirmed by expert neuropathologists were available, to serve as the training set. Using this training model, the CT regions on the remaining cohort (TCGA, CPTAC) (i.e. test set) were identified. For the survival-prediction stage, the last layer of ResNet18 model was replaced with a Cox layer (ResNet-Cox), and further fine-tuned using OS and censor information. Independent validation of ResNet-Cox model was performed on two hold-out sites from TCGA and one from CPTAC.

Results: Our segmentation model achieved an accuracy of 0.89 in reliably identifying CT regions on the validation data. The segmented CT regions on the test cohort were further confirmed by two experts who qualitatively confirmed the tumor segmentations. Our ResNet-Cox model achieved a concordance-index of 0.73 on MD Anderson Cancer Center (N=60), 0.71 on Henry Ford Hospital (N=96), and 0.68 on CPTAC data (N=41).

Conclusion: Deep-learning features captured from cellular tumor of H&E-stained histopathology images may predict survival in Glioblastoma.

### Example Use Case 2

Methods: We employed N=514 surgically resected H&E stained tissue slides obtained from multiple sites to predict sex-specific overall survival (OS) in GBM patients. Our approach contains two stages. First stage involved segmentation of tumor from non-tumor regions and background using Resnet-18 model, while the second stage leveraged the segmented tumor regions to build the sex-specific prognostic models for prediction of OS. Apart from training the sex-specific survival models, we also trained and evaluated an all-comers model using both male and female cohorts (n=264) for comparison. Additionally, we incorporated multimodal data that included clinical and molecular information of the patients along with the deep-learned H&E features to build sex-specific multi-modal ResNet-Cox model (mResNet-Cox) models. Lastly, we interpreted our trained models by visualizing the risk-density maps to illustrate the tumor microenvironment that contributed to variable risk prediction across males and females.

Findings: Sex-specific mResNet-Cox models that incorporated multimodal data such as clinical, molecular, and deep-learned H&E features of the patients yielded C-index (0.696, 0.736, 0.731, 0.729) for male cohort, while C-index (0.729, 0.738, 0.724, 0.696) for female cohort across training and three validation cohorts respectively. In order to interpret the tumor-microenvironment that contribute to the survival risk-prediction, risk-density maps were visualized where microvascular proliferation (an important hallmark of malignant progression) showed association with high-risk in both males and females. Additionally, we identified that pseudopalisading cells that promote tumor growth were associated with high-risks in males, while high tumor infiltration was associated with high-risks in females.

Interpretation: End-to-end deep learning approach using routine H&E stained slides, trained separately on male and female GBM patients, will allow for developing (a) more accurate patient-centric prognostic models of GBM tumors, and (b) capture sex-specific histological attributes of the GBM tumor microenvironment associated with high risks of poor survival.

Therefore, in some embodiments, the present disclosure relates to a non-transitory computer-readable medium storing computer-executable instructions that, when executed, cause a processor to perform operations, including obtaining an imaging data set having one or more digitized images from one or more patients having glioblastoma (GBM); utilizing a machine learning pipeline to generate a prognosis using one or more machine learning features that describe a morphology of the one or more digitized images, utilizing the machine learning pipeline including utilizing a first machine learning stage to segment the one or more digitized images to identify one or more cellular tumor (CT) regions; and utilizing a second machine learning stage to generate one or more machine learning features that describe a morphology of the one or more CT regions and to further determine the prognosis from one or more machine learning features.

In other embodiments, the present disclosure relates to a prognostic apparatus, including a memory configured to store an imaging data set having one or more digitized images from one or more patients having glioblastoma (GBM); a machine learning pipeline, including a first machine learning stage configured to receive the one or more digitized images and to segment the one or more digitized images to identify one or more cellular tumor (CT) regions; and a second machine learning stage configured to generate one or more machine learning features that describe a morphology of the one or more CT regions and to determine a prognosis of the one or more patients from one or more machine learning features.

In yet other embodiments, the present disclosure relates to a method of determining a prognosis for a patient with Glioblastoma, including providing an imaging data set having one or more digitized images from one or more patients having glioblastoma (GBM); utilizing a first machine learning stage to segment the one or more digitized images to identify one or more cellular tumor (CT) regions; and utilizing a second machine learning stage to generate one or more machine learning features describing a morphology of the one or more CT regions and to determine a prognosis from one or more machine learning features.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A non-transitory computer-readable medium storing computer-executable instructions that, when executed, cause a processor to perform operations, comprising:
obtaining an imaging data set comprising one or more digitized images from one or more patients having glioblastoma (GBM);
utilizing a machine learning pipeline to generate a prognosis using one or more machine learning features that describe a morphology of the one or more digitized images, wherein utilizing the machine learning pipeline comprises:
utilizing a first machine learning stage to segment the one or more digitized images to identify one or more cellular tumor (CT) regions; and
utilizing a second machine learning stage to generate one or more machine learning features that describe a morphology of the one or more CT regions and to further determine the prognosis from one or more machine learning features.

2. The non-transitory computer-readable medium of claim 1, wherein the first machine learning stage separates the one or more CT regions from necrotic regions or background regions.

3. The non-transitory computer-readable medium of claim 1, wherein the first machine learning stage comprises a ResNet model.

4. The non-transitory computer-readable medium of claim 1, wherein the second machine learning stage comprises a ResNet model that has one or more ResNet layers and one or more layers comprising a Cox regression model.

5. The non-transitory computer-readable medium of claim 1, wherein the second machine learning stage comprises a ResNet-18 model that has 17 ResNet layers and one layer comprising a Cox proportional-hazards model.

6. The non-transitory computer-readable medium of claim 1, wherein the operations further comprise:
separating the one or more digitized images into a male data set and a female data set;
providing the male data set to the machine learning pipeline, wherein the machine learning pipeline is configured to utilize first machine learning algorithms to predict a male prognosis from one or more machine learning features describing a morphology of the male data set; and
providing the female data set to the machine learning pipeline, wherein the machine learning pipeline is configured to utilize second machine learning algorithms to predict a female prognosis from one or more machine learning features describing a morphology of the female data set.

7. The non-transitory computer-readable medium of claim 1,
wherein the second machine learning stage comprises a ResNet-18 model that has 17 ResNet layers and one layer comprising a Cox proportional-hazards model; and
wherein the Cox proportional-hazards model is configured to receive one or more multi-modal inputs in addition to an output of the 17 ResNet layers.

8. The non-transitory computer-readable medium of claim 1, wherein the operations further comprise:
determining a risk score based upon an output of the second machine learning stage.

9. The non-transitory computer-readable medium of claim 8, wherein the operations further comprise:
generating one or more of a risk density map and a t-SNE plot based upon the risk score.

10. The non-transitory computer-readable medium of claim 1, wherein the second machine learning stage determines the prognosis from only one or more machine learning features.

11. The non-transitory computer-readable medium of claim 1, wherein the one or more digitized images respectively comprise multiple CT regions.

12. A prognostic apparatus, comprising:
a memory configured to store an imaging data set comprising one or more digitized images from one or more patients having glioblastoma (GBM);
a machine learning pipeline, comprising:
a first machine learning stage configured to receive the one or more digitized images and to segment the one or more digitized images to identify one or more cellular tumor (CT) regions; and
a second machine learning stage configured to generate one or more machine learning features that describe a morphology of the one or more CT regions and to determine a prognosis of the one or more patients from one or more machine learning features.

13. The prognostic apparatus of claim 12,
wherein the first machine learning stage comprises a first convolutional neural network (CNN) model; and
wherein the second machine learning stage comprises a ResNet model that has one or more ResNet layers and one or more layers comprising a linear regression model.

14. The prognostic apparatus of claim 12, wherein the second machine learning stage comprises a ResNet model that has a plurality of ResNet layers and one layer comprising a Cox proportional-hazards model.

15. The prognostic apparatus of claim 12, further comprising:
a separation circuit configured to separate the one or more digitized images into a male data set and a female data set; and
wherein the machine learning pipeline is further configured to:
utilize first machine learning algorithms to predict a male prognosis using one or more machine learning features describing a morphology of CT regions within the male data set; and
utilize second machine learning algorithms to predict a female prognosis using one or more machine learning features describing a morphology of CT regions within the female data set.

16. A method of determining a prognosis for a patient with Glioblastoma, comprising:
providing an imaging data set comprising one or more digitized images from one or more patients having glioblastoma (GBM);
utilizing a first machine learning stage to segment the one or more digitized images to identify one or more cellular tumor (CT) regions; and
utilizing a second machine learning stage to generate one or more machine learning features describing a morphology of the one or more CT regions and to determine a prognosis from the one or more machine learning features.

17. The method of claim 16,
wherein the first machine learning stage comprises a first convolutional neural network (CNN) model; and
wherein the second machine learning stage comprises a ResNet model that has one or more ResNet layers and one or more layers comprising a linear regression model.

18. The method of claim 16, wherein the second machine learning stage comprises a ResNet model that has a plurality of ResNet layers and one layer comprising a Cox proportional-hazards model.

19. The method of claim 16, further comprising:
separating the one or more digitized images into a male data set and a female data set;
providing the male data set to the first machine learning stage and the second machine learning stage, wherein the first machine learning stage and the second machine learning stage are configured to utilize first machine learning algorithms to predict a male prognosis from one or more machine learning features describing a morphology of CT regions within the male data set; and
providing the female data set to the first machine learning stage and the second machine learning stage, wherein the first machine learning stage and the second machine learning stage are configured to utilize second machine learning algorithms to predict a female prognosis from one or more machine learning features describing a morphology of CT regions within the female data set.

20. The method of claim 16, further comprising:
generating the prognosis based on both an output of a ResNet layer and one or more multi-modal inputs.
